(19) United States
(12) Patent Application Publication
(10) Pub. No.: US 2026/0354717 A1
(43) Pub. Date: Nov. 12, 2026

(54) SYSTEMS AND METHODS FOR MONITORING PATIENT CONDITION

(71) Applicant: NIRSense, Inc., Richmond, VA (US)

(72) Inventors: Daniel C. RODENHAVER, Richmond (VA); Thibault Pierre Thierry ROUMENGOUS DE FESTES, Richmond, VA (US); Jason Michael STROHMAIER, Richmond, VA (US); Ryan Casey BOUTWELL, Richmond, VA (US)

(73) Assignee: NIRSense, Inc., Richmond, VA (US)

(21) Appl. No.: 19/490,742

(22) PCT Filed: May 30, 2024
(86) PCT No.: PCT/US2024/031657

§ 371 Date: Dec. 5, 2025

Related U.S. Application Data
(60) Provisional application No. 63/506,749, filed on Jun. 7, 2023.

(51) Int. Cl. A61B 5/1455; A61B 5/00; A61B 5/0205; A61B 5/026; A61B 5/145

(52) U.S. Cl. CPC A61B 5/14552; A61B 5/0002; A61B 5/0205; A61B 5/14553; A61B 5/7207; A61B 5/7264; A61B 5/7282; A61B 5/0261; A61B 5/14546; A61B 5/4836; A61B 2562/0238; A61B 2562/0242; A61B 2562/164; A61B 2562/182

(57) ABSTRACT

A method may include placing a near-infrared spectroscopy (NIRS) system on a body part of the user. The method may include detecting, via the NIRS system, one or more biometric properties associated with the user. The method may include monitoring a respective change associated with at least a first biometric property of the one or more biometric properties over a predefined period of time. The method may include determining whether the respective change satisfies one or more predetermined thresholds. Responsive to determining the respective change does not satisfy the one or more predetermined thresholds, the method may include initiating one or more feedback actions.

### RELATED APPLICATIONS

This Application is a Patent Cooperation Treaty (PCT) application of, and claims priority under Article 8 of the PCT to, U.S. Provisional Patent Application No. 63/506,749, filed Jun. 7, 2023, entitled “SYSTEMS AND METHODS FOR MONITORING PATIENT CONDITION,” the entire contents of which are fully incorporated herein by reference for all purposes.

The following applications are related applications, and each is incorporated by reference in its entirety for all purposes: U.S. Provisional Patent Application No. 63/170,201, filed Apr. 2, 2021, entitled “NEAR-INFRARED SPECTROSCOPY SYSTEMS AND METHODS,” U.S. Provisional Patent Application No. 63/351,237, filed Jun. 10, 2022, entitled “SYSTEMS AND METHODS FOR DETECTING BIOMETRIC PARAMETERS,” U.S. Provisional Patent Application No. 63/356,740, filed Jun. 29, 2022, entitled “SYSTEMS AND METHODS FOR DETECTING BIOMETRIC AND NEUROPHYSIOLOGICAL PARAMETERS,” and U.S. Provisional Patent Application No. 63/389,282, filed Jul. 14, 2022, entitled “MODULATING USER BEHAVIOR AND BIOFEEDBACK USING A NEUROIMAGING AND NEUROSTIMULATING SYSTEM.”

### TECHNICAL FIELD

The present disclosure relates generally to systems and methods for monitoring patient condition. In particular, the present disclosure relates to systems and methods for detecting biometric properties of a user, and to detecting whether those properties experience respective changes over a predefined period of time.

### BACKGROUND

Near-infrared spectroscopy (NIRS) devices interrogate biological tissue using a selection of light wavelengths in the red and near-infrared (NIR) region of the electromagnetic spectrum. These wavelengths are particularly well suited for deep light penetration through tissue, versus lower wavelengths of light that are scattered or absorbed by confounding factors in the body and thus cannot reach the tissue depth of these red and NIR wavelengths. NIRS devices generally feature at least two wavelengths of light output in this range and at least one detector, and including additional optical elements can allow different depths of sensing.

Red and near-infrared wavelengths are particularly effective for non-invasively sensing different molecular states of hemoglobin in various body tissues. Unfortunately, existing NIRS devices are typically expensive, large desktop units with disintegrated sensor and processing systems. This lack of portability limits the usefulness of NIRS outside of the surgical suite, laboratory, and research environments. Some portable solutions include a sensor-only patch with wired communication to a separate portable, pocketable, or head-worn processing and communications unit. These changes represent only a nominal improvement, as the processing unit is itself not fully wearable and risks physically detaching from the sensor unit through movement or cable weight. These limitations greatly decrease the wearability and utility of such systems. These semi-ambulatory systems are also typically not designed to be used in parallel, where individual NIRS sensor systems work in tandem across the body or across a population to continually sense physiological features at multiple places using a common interface. Non-ambulatory systems can have more sensor inputs, but these are limited by the total number of ports designed into the physical system itself. Therefore, there exists a need for integrated NIRS systems and methods of using those systems to interrogate biological tissue.

### SUMMARY

In some embodiments, a method of monitoring a condition of a user is disclosed. The method may include placing a near-infrared spectroscopy (NIRS) system on a body part of the user. The method may include detecting, via the NIRS system, one or more biometric properties associated with the user. The method may include monitoring a respective change associated with at least a first biometric property of the one or more biometric properties over a predefined period of time, such as by measuring one or more metrics. The method may include determining whether the respective change satisfies one or more predetermined thresholds. Responsive to determining the respective change does not satisfy the one or more predetermined thresholds, the method may include initiating one or more feedback actions.

In some embodiments, the NIRS system may be a miniaturized, ingress-protected, crush-resistant, wireless, autonomous, and self-contained NIRS monitoring wearable system.

In some embodiments, the predefined period of time may be associated with a duration of a medical procedure and/or an observational period.

### BRIEF DESCRIPTION OF THE DRAWINGS

The accompanying drawings, which are incorporated in and form a part of the specification, illustrate the embodiments of the invention and together with the written description serve to explain the principles, characteristics, and features of the invention. In the drawings:

FIG. 1 depicts an embodiment of a system used for monitoring patient condition, in accordance with the present disclosure.

FIG. 2 depicts an embodiment of a substrate of the system of FIG. 1, in accordance with the present disclosure.

FIG. 3 depicts an embodiment of an electronics module of the system of FIG. 1, in accordance with the present disclosure.

FIG. 4 is a flowchart of a method for monitoring patient condition, in accordance with the present disclosure.

### DETAILED DESCRIPTION

This disclosure is not limited to the particular systems, devices, and methods described, as these may vary. The terminology used in the description is for the purpose of describing the particular versions or embodiments only, and is not intended to limit the scope of the disclosure.

The following terms shall have, for the purposes of this application, the respective meanings set forth below. Unless otherwise defined, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention.

As used herein, the singular forms “a,” “an,” and “the” include plural references, unless the context clearly dictates otherwise. Thus, for example, reference to a “fiber” is a reference to one or more fibers and equivalents thereof known to those skilled in the art, and so forth.

As used herein, the term “about” means plus or minus 10% of the numerical value of the number with which it is being used. For example, about 50 mm means in the range of 45 mm to 55 mm.

As used herein, the term “consists of” or “consisting of” means that the device or method includes only the elements, steps, or ingredients specifically recited in the particular claimed embodiment or claim.

In embodiments or claims where the term “comprising” is used as the transition phrase, such embodiments can also be envisioned with replacement of the term “comprising” with the terms “consisting of” or “consisting essentially of.”

It is to be understood that the mention of one or more method steps does not preclude the presence of additional method steps or intervening method steps between those steps expressly identified. It is also to be understood that the listing of one or more method steps in any order does not preclude the performance of such one or more methods steps in alternative orders, nor does it preclude the simultaneous performance of any such one or more method steps. Similarly, it is also to be understood that the mention of one or more components in a device or system does not preclude the presence of additional components or intervening components between those components expressly identified.

It will be understood by those within the art that, in general, terms used herein are generally intended as “open” terms (for example, the term “including” should be interpreted as “including but not limited to,” the term “having” should be interpreted as “having at least,” the term “includes” should be interpreted as “includes but is not limited to,” et cetera). While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups.

In addition, even if a specific number is explicitly recited, those skilled in the art will recognize that such recitation should be interpreted to mean at least the recited number (for example, the bare recitation of “two components,” without other modifiers, means at least two components, or two or more components). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, sample embodiments, or drawings, should be understood to contemplate the possibilities of including one of the terms, either of the terms, or both terms. For example, the phrase “A or B” will be understood to include the possibilities of “A” or “B” or “A and B.” Furthermore, where features of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, et cetera. As a non-limiting example, each range discussed herein can be readily broken down into a lower third, middle third and upper third, et cetera. As will also be understood by one skilled in the art all language such as “up to,” “at least,” and the like include the number recited and refer to ranges that can be subsequently broken down into subranges as discussed above. Finally, as will be understood by one skilled in the art, a range includes each individual member. Thus, for example, a group having 1-3 components refers to groups having 1, 2, or 3 components. Similarly, a group having 1-5 components refers to groups having 1, 2, 3, 4, or 5 components, and so forth.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

Near-infrared spectroscopy devices interrogate biological tissue using a selection of light wavelengths in the red and NIR region of the electromagnetic spectrum. These wavelengths are particularly well suited for deep light penetration through tissue, versus other wavelengths of light that are scattered or absorbed by confounding factors in the body and thus cannot reach the tissue depth of these red and NIR wavelengths. NIRS devices generally feature at minimum two wavelengths of light output in this range and at least one detector, and including additional optical elements can allow different depths of sensing.

Red and near-infrared wavelengths are particularly effective for non-invasively sensing different molecular states of hemoglobin in various body tissues. Hemoglobin is a strong absorber of light in the middle of the visible light spectrum but has a low optical extinction coefficient within the higher wavelengths of the visible range. Within the NIR wavelengths, for hemoglobin's oxygenation states, deoxy-and oxyhemoglobin's absorption spectra cross at an isosbestic point near 805 nm, allowing NIRS systems to differentiate oxygenation states of hemoglobin using light sources above and below this wavelength. With this differentiation, NIRS can be used for a variety of sensing mechanisms related to the body's circulatory, pulmonary, neurological, and other functional systems.

Hemoglobin also allows for binding of ligands other than oxygen. These other molecular states of hemoglobin, such as carboxyhemoglobin and methemoglobin, have unique optical absorption characteristics in the NIR range. Investigating these molecular states can elucidate competitive binding and indicate histologic changes in tissue oxygenation such as tissue poisoning. Hemoglobin has a competitive binding efficiency for many molecules, such as carbon monoxide (CO), cyanide (CN—), sulfur monoxide (SO), sulfide (S2—), and others in these groups. Nitric oxide (NO) also binds to hemoglobin and can be detected optically. Investigating the NIR spectra of these additional bound states of hemoglobin can indicate tissue status and toxicity by inhibiting oxygen binding as well as enable sophisticated physiological monitoring of body systems.

NIRS systems may calculate oxygenation levels using the modified Beer-Lambert law (mBLL), which only requires one bank of light sources. Using the mBLL offers the translation of raw optical signals into actionable oxygenation details. Alternatively, NIRS systems may employ spatially resolved spectroscopy (SRS), which can use both short-and long-distance measurements. Separately, short channel information can be subtracted from long channel information to more accurately isolate, for example, brain activity and the contributions from internal (e.g., cerebral) vasculature and external (e.g., skin) vasculature. In certain systems with the appropriate engineering design and algorithm capabilities, tissue perfusion measured on the shorter channels may also be compared to tissue perfusion measured on the deeper channels in order to characterize changes in tissue oxygenation at multiple depths, either on different body locations or in the same body location.

Unfortunately, existing NIRS devices are typically expensive, large desktop units with disintegrated sensor and processing systems. This lack of portability limits the usefulness of NIRS outside of the surgical suite, laboratory, and research environments. Even in such controlled environments, these devices sometimes fail because they are difficult to integrate into a user's system when the planned testing involves any form of motion.

Some portable solutions include a sensor-only patch with wired communication to a separate portable, pocketable, or head-worn processing and communications unit. These changes represent only a nominal improvement, as the processing unit is itself not fully wearable and risks physically detaching the sensor unit through movement or cable weight. These limitations greatly decrease the wearability and utility of such systems. These semi-ambulatory systems are also typically not designed to be used in parallel, where individual NIRS sensor systems work in tandem across the body or across a population to continually sense physiological features at multiple places using a common interface. Non-ambulatory systems can have more sensor inputs, but these are limited by the total number of ports designed into the physical system itself. Therefore, there exists a need for integrated NIRS systems and methods of using those systems to interrogate biological tissue.

Further, existing tissue oximeters can be used to continuously and non-invasively estimate tissue oxygenation (StO2) using NIRS technology. Photoplethysmography (PPG) monitors periodic oscillations of reflected light from the pulsation of arterial blood to continuously estimate arterial oxygen saturation through pulse oximetry (SpO2), as well as pulse rate (PR), respiratory rate (RR), perfusion, blood pressure, and other metrics using red and near infrared optical components. Pulse oximetry (SpO2) provides a systematic biomarker (i.e., not tissue-specific) of arterial blood oxygenation, while StO2 can provide mixed venous oxygenation at specific body sites (e.g., cerebral oxygenation, muscle oxygenation, and other specific organs).

In addition, existing monitors, such as heart rate monitors, can only monitor electrical activity, as opposed to being able to determine whether the heart is moving blood properly, or to differentiate between a heart versus, for example, a lung problem.

Accordingly, the systems and methods disclosed herein may provide for the measurement of tissue oxygen in combination with other measurements, such as electrocardiography (ECG) for cardiac electrophysiology (e.g., heart timing), electromyography (EMG) for muscle firing, electroencephalography (EEG) for cerebral electrical activity, electrooculography (EOG) for eye movements, ultrasound for monitoring flow rates (e.g., ultrasound doppler), or other types of measurements. The systems and methods disclosed herein may further provide wearable, ruggedized NIRS systems configured to enable the continuous and remote monitoring of patient physiology in both traditional and non-traditional healthcare environments, including in primary care clinics, at-home environments, remote environments, and austere and low medical resource environments, in order to improve care provider decision-making by providing physiological data currently unavailable through use of existing systems and designs. The systems and methods disclosed herein may provide for the monitoring of neurological function for various medical applications, such as trauma, disease, injury, etc. The ability to continuously monitor changes in blood oxygenation in shallow and deeper tissues, combined with the monitoring of PR, RR, and perfusion, can enable a transformational capability to extend medical care outside of centralized brick-and-mortar facilities and opens the potential for expanded medical decision-making capabilities with data that is currently unavailable to medical practitioners today. The networked aspect of the NIRS systems disclosed herein also allows for monitoring of multiple patients in different locations and for monitoring multiple ambulatory patients in one location, enabling the potential for group monitoring in a range of physiological states and environments.

Data can be recorded either as an instantaneous measure of physiology (e.g., SpO2 level, StO2 level, etc.) or as a longitudinal tracker of changing physiology over time (e.g., StO2 or SpO2 trend monitoring). A point measure can be made over successive time periods to track a patient's evolution of physiology, for example, during regular physician checkups. The duration of monitoring time may be on the order of minutes, such as in ischemia-reperfusion assessments using vascular occlusion testing related to blood pressure measurement, in monitoring orthostatic responses in physiology such as reaction to standing, in monitoring cardiopulmonary function during a standardized mild physical activity, such as a six-minute walk test, in dosing physical therapy, in titrating pharmacological interventions, or in other medical, health, or performance interventions. The duration may be on the order of hours, such as sleeping overnight without orthostatic changes or during the onset of infection or rapid progression of diseases affecting tissue oxygenation, such as compartment syndrome. The duration may be on the order of days, such as monitoring changes in cardiopulmonary or vascular or muscle function postoperatively, or weeks, such as for preoperative patients at risk of degradation or “pre-habilitating” for an upcoming surgery. Patients may be monitored on the order of months to track long period evolutions in physiological fitness, disease progression, injury recovery, or other aspects. Patients may also be monitored over longer periods of time in order to detect transient effects in tissue oxygenation kinetics related to their physiological status, such as in exertional compartment syndrome, cognitive workload, and fatigue, stress, and other causes with temporary effects.

Patients can even be monitored in multiple locations (e.g., anatomical locations, geographic locations) and/or at the same time using the systems and methods disclosed herein, from which data may be collected to indicate degradation in local tissue health when compared to systemic measures.

The data may be visible to the patient, such as by a monitor on a wearable device or by a paired auxiliary device running visualization software (e.g., a phone/tablet application, a base station, a connected laptop/desktop computer, etc.). Such device might also analyze and present additional data to the user. The data may also be visible to an attending care provider, or to a remote care provider over a telemedicine or similar network. Such data may be combined with other physiological data (e.g., collected via the systems and methods disclosed herein, or by a remote system) to build a comprehensive image of patient status.

In some embodiments, the NIRS systems disclosed herein may be configured to measure three or more wavelengths at three or more optical depths, as further discussed herein. In some embodiments, the NIRS systems disclosed herein may weigh approximately 2.0 ounces or less (e.g., approximately 2.1 ounces, 1.9 ounces, 1.6 ounces, 1.4 ounces, 1.2 ounces, 1.0 ounces, 0.8 ounces, 0.6 ounces, 0.4 ounces, 0.2, ounces, etc.). In some embodiments, the NIRS systems disclosed herein may have a volume of approximately 30 cm³ or less (e.g., approximately 28.5 cm³, 26.0 cm³, 22.0 cm³, 20.0 cm³, 17.0 cm³, 15.5 cm³, 13.0 cm³, 9.0 cm³ , 7.5 cm³, 4.0 cm³, 2.5 cm³ , etc.). In some embodiments, the NIRS systems disclosed herein may have a battery life of at least approximately 12.0 hours.

In some embodiments, the NIRS systems disclosed herein may have the ability to adapt their gain and performance based on location, need, and/or environment.

In some embodiments, the systems disclosed herein may incorporate one or more of the systems, devices, and/or components as described in U.S. Provisional Application Nos. 63/170,201, 63/351,237, 63/356,740, and 63/389,282, which are each incorporated herein by reference in their entirety, and portions of which are included herein as Appendices to the present application.

Reference will now be made in detail to example embodiments of the disclosed technology that are illustrated in the accompanying drawings and disclosed herein. Wherever convenient, the same reference numbers will be used throughout the drawings to refer to the same or like parts.

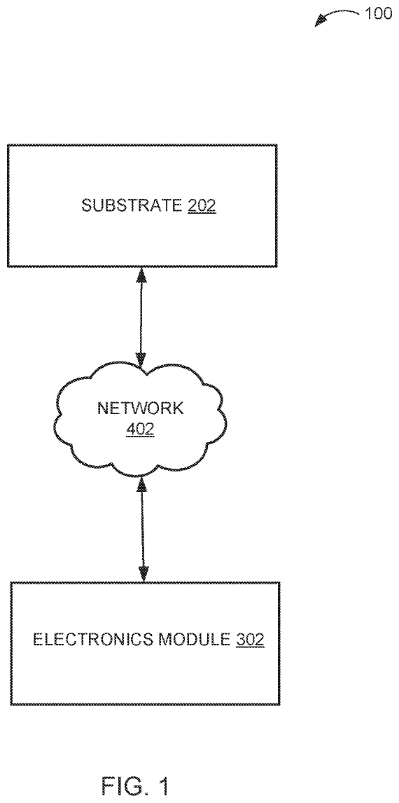

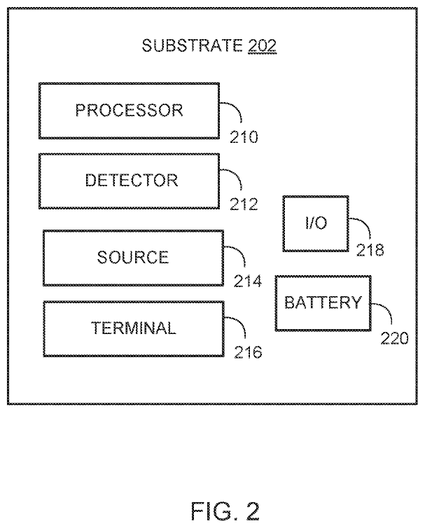

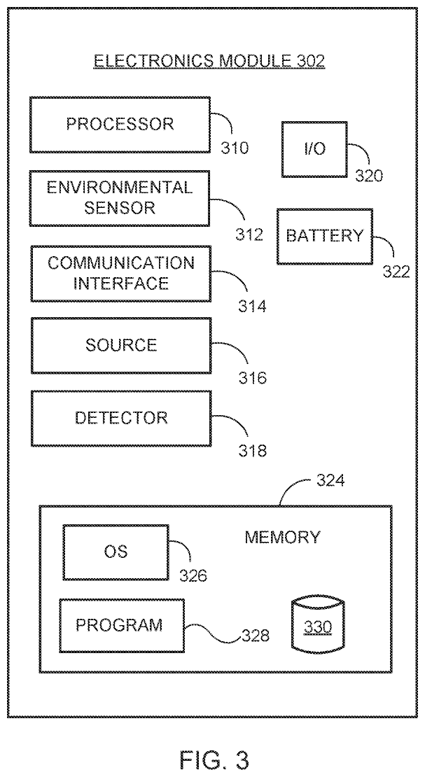

FIG. 1 depicts an embodiment of a system 100 used for monitoring patient condition, in accordance with the present disclosure. The system 100 may include a substrate 202 configured to communicate with an electronics module 302 over a network 402, as further discussed below with respect to FIGS. 2-3.

FIG. 2 depicts an embodiment of a substrate 202 used for monitoring patient condition, in accordance with the present disclosure. In some embodiments, the substrate 202 may be configured to be placed on a body part of a user (e.g., a patient). For example, substrate 202 may be coupled to (e.g., placed on top of or embedded within) a flexible material, such that substrate 202 may be placed (e.g., with an adhesive) on a body part of a user and conform to the shape of that body part (e.g., a forehead, sternum, leg, etc.). In some embodiments, the substrate 202 and/or the flexible material may also provide for electrical insulation and/or optical isolation and/or thermal isolation and/or acoustic isolation of any NIRS and/or PPG optics, and/or any electrical connection lines to electrodes, thermal sensors, or ultrasound transducers mounted on substrate 202.

In some embodiments, the substrate 202 may include one or more materials, such as silicone, nylon, epoxy, a bioinert polymer, a biocompatible polymer, a woven or nonwoven textile, an adhesive film, a flexible circuit board, flexible sensors and electronics, or a combination thereof. In some embodiments, the substrate 202 and any components mounted onto the substrate 202 may be configured to provide mechanical flexibility, allowing the substrate 202 to conform to and/or adhere to a surface. In some embodiments, the substrate 202 may be configured to be integrated into clothing or other equipment designed to be worn or applied to a user.

In some embodiments, the shape and/or dimensions of the substrate 202 may be different depending on the specific patient and/or use case. Additionally, substrate 202 may also be configured of any size.

As shown in FIG. 2, the substrate 202 may include a processor 210, a sensor or detector 212, a source 214 (e.g., a light source, an electrode, an ultrasound transducer), a terminal 216, an input/output (I/O) device 218, and/or a power source 220 (e.g., a battery) configured to power the substrate 202.

Detector 212 may be configured to sense or detect oximetry parameters of a user, such as SpO2 and StO2. Detector 212 may be configured to detect these parameters at various tissue depths associated with the applicable body part, as discussed herein.

The I/O device 218 may be configured to connect the substrate 202 to one or more other components of system 100 or one or more components external to system 100, such as a computing device (e.g., a laptop or other “smart” device).

The source 214 may include a single light source. In other embodiments, the source 214 may include multiple light sources, such as 2 light sources, 3 light sources, 4 light sources, 5 light sources, and so on. In some embodiments, each light source may include one or more light emitting diodes (LEDs). In some embodiments, each source may include a single tunable light source such as a broadband LED coupled with a miniature monochromator. In some embodiments, each source may include one or more laser diodes. In an embodiment, the source 214 may include a light source driver capable of selecting between the different light sources or selecting a wavelength from a tunable light source. In some embodiments, the source 214 may include ultrasound or electrode sources.

In some embodiments, the source 214 may be capable of emitting a first set of wavelengths of red or near-infrared light. In some embodiments, each light source within the source 214 may be capable of independently emitting a wavelength. The first set of wavelengths may comprise 1 wavelength, 2 wavelengths, 3 wavelengths, 4 wavelengths, 5 wavelengths, 6 wavelengths, 7 wavelengths, 8 wavelengths, 9 wavelengths, 10 wavelengths, or any other number of wavelengths known in the art. In some embodiments, each wavelength within the first set of wavelengths may independently be from about 650 nm to about 950 nm. Each wavelength may be, for example, about 650 nm, about 655 nm, about 660 nm, about 665 nm, about 670 nm, about 675 nm, about 680 nm, about 685 nm, about 690 nm, about 695 nm, about 700 nm, about 705 nm, about 710 nm, about 715 nm, about 720 nm, about 725 nm, about 730 nm, about 735 nm, about 740 nm, about 745 nm, about 750 nm, about 755 nm, about 760 nm, about 765 nm, about 770 nm, about 775 nm, about 780 nm, about 785 nm, about 790 nm, about 795 nm, about 800 nm, about 805 nm, about 810 nm, about 815 nm, about 820 nm, about 825 nm, about 830 nm, about 835 nm, about 840 nm, about 845 nm, about 850 nm, about 855 nm, about 860 nm, about 865 nm, about 870 nm, about 875 nm, about 880 nm, about 885 nm, about 890 nm, about 895 nm, about 900 nm, about 905 nm, about 910 nm, about 915 nm, about 920 nm, about 925 nm, about 930 nm, about 935 nm, about 940 nm, about 945 nm, about 950 nm, or any range between any two of these values, including endpoints. In some embodiments, each wavelength within the first set of wavelengths may be greater than about 805 nm. In some embodiments, the average of the first set of wavelengths may be greater than about 805 nm. In certain embodiments, the first set of wavelengths may include five individual wavelengths to interrogate the targeted tissue: one in the red region below 730 nm, one in the NIR region below the 805 nm isosbestic point, one near or at the 805 nm isosbestic point, and two in the NIR region above the isosbestic point.

Turning to FIG. 3, system 100 may include one or more electronics modules 302. Each electronics module 302 may be configured with the same or similar components; however, in some embodiments, different electronics modules 302 may be configured to detect and process different types of parameters of a user. For example, a first electronics module 302 may be configured to detect and process optical biometric properties of a user, while a second electronics module 302 may be configured to detect and process non-optical biometric properties of a user. In some embodiments, system 100 may include a single electronics module 302 that may include the hardware and functionality of a first and second electronics module 302, and may be configured to carry out the program instructions for both electronics modules within a single, integrated package.

In some embodiments, electronics module 302 may be communicatively coupled to the substrate 202 (e.g., via network 402, as shown in FIG. 1), and may include a processor 310, a memory device 324, and instructions stored on the memory device 324 that may direct the processor 310 to perform one or more actions. The electronics module 302 may be configured to receive a signal from one or more detectors 212 mounted on the substrate 202, and to process the received signal based on one or more predefined algorithms. Based on the processed signal, the electronics module 302 may be configured to determine whether the user, e.g., on or to which substrate 202 is coupled, requires one or more feedback actions, as further discussed below.

As shown in FIG. 3, the electronics module 302 may further include one or more environmental sensors 312, a communication interface 314, a light source 316, a detector 318, an I/O device 320, and/or an energy storage device 322 (e.g., a battery) configured to power the electronics module 302. Memory device 324 may include an operating system (OS) 326 and program 328, and a database 330. Operating system 326 may be a real-time operating system (RTOS) or program instructions in system firmware operating on the processor 310. Program 328 may include stored instructions that direct the processor 310 to perform one or more steps toward processing signals received from substrate 202, as discussed herein.

In some embodiments, the processor 310 may be configured to select emitted set(s) of wavelengths, as discussed above, and respective distance(s) of the source 214 from the detector 212. The input parameter may include, for example, a temperature, a lighting condition, a velocity, an acceleration, a change in acceleration, a pressure, a change in pressure, a volume, a change in volume, a measurement made, recorded, or calculated by the system, a communication from another device or system, or a combination thereof.

In some embodiments, the environmental sensor(s) 312 can measure parameters surrounding the patient and not the patient directly. Environmental properties may include, for example, temperature, humidity, pressure, motion, chemical composition, ambient light intensity, sound, etc., of the external environment in which the patient is positioned. For example, if the patient's body temperature is calculated as being too high, such as above some predetermined threshold, the electronics module 302, e.g., via the processor 310, may be configured to adjust a thermostat located in the room in which the patient is located. As another example, environmental sensor(s) 312 may include a microphone configured to receive spoken instructions informing the electronics module 302 how to operate.

In some embodiments, the communication or connection interface 314 (which may also or instead be included on substrate 202) can facilitate connections that can be, for example, a wireless connection, a wired connection, a Bluetooth connection, a near-field communication (NFC) connection, a radio frequency identification (RFID) connection, or a combination thereof. In some embodiments, data processing and real-time feedback may occur within the components onboard the substrate 202, or offboard through communication with an external computing device, as discussed below. The external computing device may comprise, for example, a smartphone, a charging or communications base station, a display screen, a tablet, a computer, a mobile or web-based application, or another device.

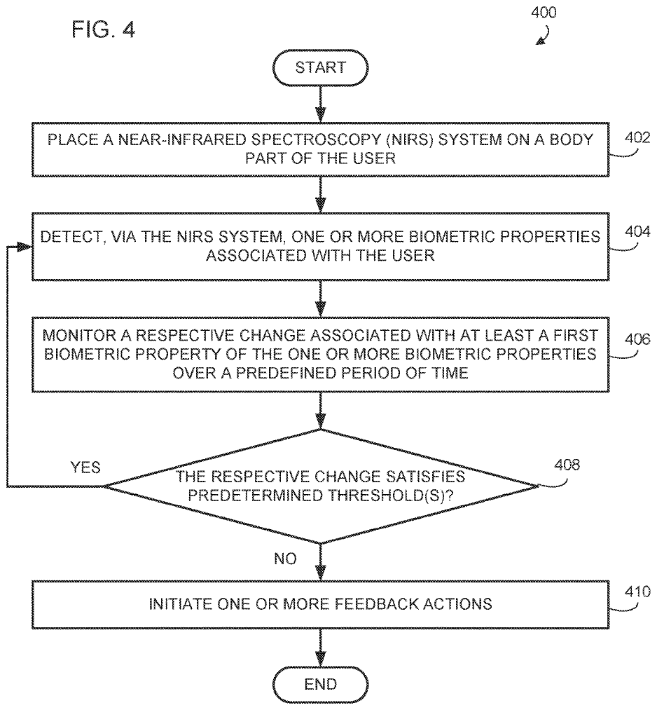

FIG. 4 is a flowchart of a method 400 for monitoring patient condition, in accordance with the present disclosure. Method 400 may be conducted using one or more components of system 100, such as substrate 202, electronics module 302, or any of the devices or systems discussed herein. It should be understood that certain embodiments of the disclosed technology may omit one or more blocks as being optional.

In block 402, the method may include placing a NIRS system on a body part of a user, e.g., a patient. The NIRS system may be any of the devices or systems as discussed herein. For example, the NIRS system may include a sensor (e.g., detector 212 of substrate 202) configured to detect one or more biometric properties of the body part and/or the user. The NIRS system may further include a non-transitory memory device (e.g., memory 324 of electronics module 302) configured to receive data from the sensor, the data corresponding to the one or more biometric properties, as further discussed below.

In some embodiments, the NIRS system may be a miniaturized, ingress-protected, crush-resistant, and/or wireless system. The NIRS system may be configured to be rugged enough such that it can withstand patient motion, movement, bumps, etc., as further discussed below. In some embodiments, the NIRS system may be autonomous and/or self-contained, such that the system may have the capability for on-board processing and/or user alerts. In some embodiments, the NIRS system may be a waterproof or water-resistant system such that it may be used in water environments, such as diving. In some embodiments, the NIRS system may have no internal voids such that it can be used in environments with both high and low ambient pressure extremes.

In some embodiments, the body part of the user may be an individual body part, such as the sternum, forehead, arm, leg, torso, etc., or may include multiple body parts.

In block 404, the method may include detecting, via the NIRS system, one or more biometric properties associated with the user. In some embodiments, the system may be configured to detect the biometric properties associated with the user's entire body, providing a systemic effect, or individual body parts, providing a local effect. In some embodiments, the biometric properties may include tissue oxygenation, ECG, EMG, EEG, EOG, PR, heart rate, RR, perfusion, blood pressure, blood flow, blood volume, cardiac output, cardiac stroke volume, cardiac ejection fraction, macrovascular and microvascular hemodynamic coherence, and/or core body temperature.

In block 406, the method may include monitoring a respective change associated with at least a first biometric property of the one or more biometric properties over a predefined period of time. In some embodiments, monitoring the respective change may involve measuring one or more metrics from the user and/or one or more predetermined thresholds from the user or the user's environment. In some embodiments, the one or more metrics may be associated with signal amplitude, rate, frequency, modulation depth, and/or recovery height, and change rates both of these individual metrics and of ratios and relations between them. In some embodiments, the one or more predetermined thresholds may be associated with oxygenation level, a blood pressure reading, a vascular occlusion test output, an ambient light exposure reading, a user motion, a sleep stage, and/or an amount of time.

In some embodiments, the predefined period of time may be associated with a duration of a medical procedure, including an examination, testing, a pre-surgical medical observation period, a post-surgical medical observational period, a blood pressure test, a vascular occlusion test, a standardized physical activity test, an ortho-static test, a hyperreactivity to standing (HRS) test, a physical therapy dosing, a medication dosing, a sleep test, a resuscitation, a defibrillation, a surgical preparation, a disease progression, and/or an injury recovery.

In some embodiments, the method may include transmitting the detected biometric properties and/or the respective change to a remote device associated with a medical data system, for example, such that a medical provider may provide medical assistance to a user, as further discussed below. The use of remote patient monitoring may provide for detection of, for example, degradation related to heart failure, lung diseases, neurological discases, injuries, or infection. In addition, it may allow for remote providing of muscle therapy, physical therapy, or emergency medicine triage.

In some embodiments, a system or device as disclosed herein may be placed on a user while at a medical appointment, such as in a physician's office. A device may be placed on a body part of the user, such as the user's sternum. The physician may then be able to monitor biometric properties of the user as the user performs certain tests in the physician's office (or other type of office, such as urgent care, a clinic, etc.), such as a blood pressure test or six-minute walk test, or after the patient leaves the physician's office such that the physician can continue to remotely monitor the patient, such as when the patient is sleeping, moving, getting in and out of the shower, exercising, getting up and down, changing position, etc. In such instances where a physician might be remotely monitoring the patient via the device, the device may be connected to a service, e.g., a phone, such that the physician may easily monitor the patient, for example, to prevent the patient from having to be admitted to a hospital. This type of remote monitoring may further enable emergency application of, for example, CPR or defibrillation, or the addition of a non-invasive perfusion measure to the aortic stenosis screening algorithm, in which case the physician might be able to implement some form of therapy, e.g., a change in patient medication or change in delivery of implanted medical device therapies.

In some embodiments, the respective change being monitored may be associated with a change in tissue oxygen while a patient is getting a blood pressure test. For example, a healthy patient would have a higher (healthier) rate of reperfusion compared to an older or sicklier person, whose rate of perfusion would come back more slowly following cuff pressure abatement. For example, a specific slower timed rate of reperfusion after a blood pressure measurement could be used as a proxy to indicate an onset of sepsis, or to classify patients into different stages or risk categories for cardiovascular or pulmonological diseases. Muscle oxygen might also be read in this fashion, for example, to bin patients into categories based on rate of reperfusion in healthy, injured, or rehabilitating patients.

In some embodiments, the respective change being monitored may be associated with a change in tissue oxygen during a six-minute walk test. For example, the NIRS device may be placed on the patient, such as on the patient's sternum, such that one or more metrics, as discussed herein, may be monitored while the patient performs the six-minute walk test.

In some embodiments, the respective change being monitored may be associated with an HRS test. For example, the NIRS device may be placed on the patient, such as on the patient's sternum, such that a patient or other provider may monitor how the sternum changes when the patient goes from a sitting to standing position. The physician or provider could then determine whether the patient is experiencing a typical drop in blood pressure.

In some embodiments, the respective change being monitored may be associated with a blood pressure change at different locations or sites on the patient's body. This could provide for the implementation of an ankle brachial index (ABI) that is informed by changes in tissue oxygenation kinetics by monitoring a patient in multiple anatomical locations simultaneously.

In some embodiments, the respective change being monitored may be associated with a change in the patient's peripheral vascular system, for example, to determine whether the patient might have peripheral vascular disease.

In block 408, the method may include determining whether the respective change satisfies one or more predetermined thresholds. In some embodiments, the one or more predetermined thresholds may be associated with oxygenation level, a blood pressure reading, a vascular occlusion test output, an ambient light exposure reading, a user motion, a sleep stage, an amount of time, or previous user physiological states measured and stored by the NIRS device or input into the NIRS device by a device user.

In block 410, responsive to determining the respective change does not satisfy the one or more predetermined thresholds, the method may include initiating one or more feedback actions.

In some embodiments, the feedback action(s) may involve adaptation of the system itself, such as making a change to a sensing parameter, recording parameter, operating parameter, etc., of the system. For example, the system may be an adaptive system wherein the system can determine that some parameter should be changed, such as the brightness of the source 214, the sensitivity of the detector 212, a sampling frequency, an alert threshold, the predetermined algorithms being used, etc.

In some embodiments, the feedback action(s) may involve transmitting or otherwise providing the processed data to a remote system (e.g., associated with a medical data system) or user, such as a caregiver or provider (e.g., a physician) of the user such that the caregiver or provider may provide, for example, medical advice or monitoring for the user.

In some embodiments, the feedback action(s) may involve taking another biometric property reading. In some embodiments, the feedback action(s) may involve providing the user with one or more types of stimulation. For example, the system may include one or more terminals (e.g., terminal 216 of substrate 202) capable of providing a user with the one or more types of stimulation, which may include one or more of transcranial direct current stimulation (tDCS), transcranial alternating current stimulation (tACS), vagus nerve stimulation (nVNS), transcutaneous electrical nerve stimulation (TENS), transcranial magnetic stimulation (TMS), ultrasound stimulation, optical stimulation, mechanical stimulation, or other types of invasive or non-invasive stimulation.

In some embodiments, the feedback action(s) may be provided to the user by changes in a real or virtual environment of the user (e.g., via a virtual reality device), by pharmacological changes (e.g., through intravascular, intramuscular, intraosseous, and/or other routes), through changes in notifications to a caregiver (as discussed above), and/or via an environmental property. The environmental property may include one or more of temperature, pressure, chemical composition, sound (e.g., continuous sound waves with changing pitch), light, motion, smell, feel, taste, or combinations thereof.

In some embodiments, the system 100 may further include an external computing device including a memory and a computer processor. The external computing device may be connected to at least a portion of at least one of the processor and the memory device via a connection, wherein at least a portion of the program instructions is also stored on the external computing device.

In some embodiments, the systems disclosed herein can be networked for concurrent monitoring of different physiological conditions of a user, the same or different physiological conditions at different locations on the body of a user, one or more physiological conditions of a group of wearers in a population, or a combination thereof.

In some embodiments, the systems and methods described herein include independent wireless devices communicating oximetry information about different areas of tissue (e.g., the brain) simultaneously. In some embodiments, the systems and methods described herein include scanning a single device over different areas of the body and continuously imaging tissue, changing methods based on determined tissue state or changes in patient condition.

In some embodiments, the systems and methods described herein include two or more independent systems that can simultaneously interrogate multiple areas of cerebral and somatic tissue to interrelate physiological status (for example, tissue oxygenation) in each area. These areas may have significantly different oxygenation signatures at any given time and simultaneously sampling these is particularly important to understand situations of local or central fatigue or recovery onset by the user. Simultaneous imaging of different body systems can also elucidate generalized physiological condition, for instance indicating systemic response to exogenous conditions such as carbon monoxide poisoning or endogenous conditions such as hemorrhage. The independently sampled processed data from each area of the body may then send signals to a user interface if a specific tissue level, condition, or status is reached, or stream data to the external processing module for real-time interpretation, or both.

In some embodiments, the NIRS systems and methods described herein include independent wireless devices communicating multi-point physiological information (e.g., oxygenation) about the brain and body simultaneously.

In some embodiments, the systems and methods described herein include multiple systems that can be worn by multiple different individuals whose data is integrated to form a comprehensive image of a group of individuals' health. This integration can be simultaneous for co-located users or asynchronous for disparate groups, or another combination. For example, comparing real-time physiological monitoring across multiple individuals can enable population monitoring and a more holistic image of group performance and wellness. Such continuous imaging can identify early threats or enhancements and increase risk or opportunity for better group performance and outcome.

Where any component discussed herein is implemented in the form of firmware and/or software, any one of a number of programming languages may be employed such as, for example, C, C++, C #, Objective C, Java®, JavaScript®, Perl, PHP, Visual Basic®, Python®, Ruby, Flash®, or other programming languages. A number of firmware and/or software components are stored in the memory and are executable by the processor. In this respect, the term “executable” means a program file that is in a form that can ultimately be run by the processor. Examples of executable programs can be, for example, a compiled program that can be translated into machine code in a format that can be loaded into a random access portion of the memory and run by the processor, source code that can be expressed in proper format such as object code that is capable of being loaded into a random access portion of the memory and executed by the processor, or source code that can be interpreted by another executable program to generate instructions in a random access portion of the memory to be executed by the processor, etc. An executable program can be stored in any portion or component of the memory including, for example, random access memory (RAM), read-only memory (ROM), hard drive, solid-state drive, USB flash drive, memory card, optical disc such as compact disc (CD) or digital versatile disc (DVD), floppy disk, magnetic tape, or other memory components.

The memory is defined herein as including both volatile and nonvolatile memory and data storage components. Volatile components are those that do not retain data values upon loss of power. Nonvolatile components are those that retain data upon a loss of power. Thus, the memory can include, for example, random access memory (RAM), read-only memory (ROM), hard disk drives, solid-state drives, USB flash drives, memory cards accessed via a memory card reader, floppy disks accessed via an associated floppy disk drive, optical discs accessed via an optical disc drive, magnetic tapes accessed via an appropriate tape drive, and/or other memory components, or a combination of any two or more of these memory components. In addition, the RAM can include, for example, static random access memory (SRAM), dynamic random access memory (DRAM), or magnetic random access memory (MRAM) and other such devices. The ROM may comprise, for example, a programmable read-only memory (PROM), an erasable programmable read-only memory (EPROM), an electrically erasable programmable read-only memory (EEPROM), or other like memory device.

Also, the processor can represent multiple processors and/or multiple processor cores and the memory can represent multiple memories that operate in parallel processing circuits, respectively. In such a case, the local interface can be an appropriate network that facilitates communication between any two of the multiple processors, between any processor and any of the memories, or between any two of the memories, etc. The local interface can include additional systems designed to coordinate this communication, including, for example, performing load balancing. The processor can be of electrical or of some other available construction.

Although some of the processing systems described herein can be embodied in software or code executed by general purpose hardware as discussed above, as an alternative the same can also be embodied in dedicated hardware or a combination of software/general purpose hardware and dedicated hardware. If embodied in dedicated hardware, each can be implemented as a circuit or state machine that employs any one of or a combination of a number of technologies. These technologies can include, but are not limited to, discrete logic circuits having logic gates for implementing various logic functions upon an application of one or more data signals, application specific integrated circuits (ASICs) having appropriate logic gates, field-programmable gate arrays (FPGAs), or other components, etc.

It should be understood that any logic or application described herein that incorporates software or code can be embodied in any non-transitory computer-readable medium for use by or in connection with an instruction execution system such as, for example, a processor in a computer system or other system. In this sense, the logic can include, for example, statements including instructions and declarations that can be fetched from the computer-readable medium and executed by the instruction execution system. In the context of the present disclosure, a “computer-readable medium” can be any medium that can contain, store, or maintain the logic or application described herein for use by or in connection with the instruction execution system. The computer-readable medium can incorporate any one of many physical media such as, for example, magnetic, optical, or semiconductor media. More specific examples of a suitable computer-readable medium include, but are not limited to, magnetic tapes, magnetic floppy diskettes, magnetic hard drives, memory cards, solid-state drives, USB flash drives, or optical discs. Also, the computer-readable medium can be a random access memory (RAM) including, for example, static random access memory (SRAM) and dynamic random access memory (DRAM), or magnetic random access memory (MRAM). In addition, the computer-readable medium can be a read-only memory (ROM), a programmable read-only memory (PROM), an erasable programmable read-only memory (EPROM), an electrically erasable programmable read-only memory (EEPROM), or other type of memory device.

Further, any logic or application described herein can be implemented and structured in a variety of ways. For example, one or more applications described can be implemented as modules or components of a single application. Further, one or more applications described herein can be executed in shared or separate computing devices or a combination thereof or to execute shared or separate components of signal analysis and feedback. For example, a plurality of the applications described herein can execute in the same computing device, or in multiple computing devices in the same computing environment. Additionally, it is understood that terms such as “application,” “service,” “system,” “engine,” “module,” and so on may be interchangeable and are not intended to be limiting.

While various illustrative embodiments incorporating the principles of the present teachings have been disclosed, the present teachings are not limited to the disclosed embodiments. Instead, this application is intended to cover any variations, uses, or adaptations of the present teachings and use its general principles. Further, this application is intended to cover such departures from the present disclosure that are within known or customary practice in the art to which these teachings pertain.

In the above detailed description, reference is made to the accompanying drawings, which form a part hereof. In the drawings, similar symbols typically identify similar components, unless context dictates otherwise. The illustrative embodiments described in the present disclosure are not meant to be limiting. Other embodiments may be used, and other changes may be made, without departing from the spirit or scope of the subject matter presented herein. It will be readily understood that various features of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular embodiments described in this application, which are intended as illustrations of various features. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

1-21. (canceled)

22. A method of monitoring a condition of a user, the method comprising:
placing a miniaturized, ingress-protected, crush-resistant, wireless, autonomous, and self-contained near-infrared spectroscopy (NIRS) monitoring wearable system on a body part of the user, wherein the NIRS system comprises a flexible substrate configured to conform to the body part and provide optical isolation of NIRS optics;
detecting, via the NIRS system, one or more biometric properties associated with the user using spatially resolved spectroscopy (SRS) with both short-distance and long-distance measurements to isolate cerebral activity contributions from skin vasculature contributions;
monitoring a respective change associated with at least a first biometric property of the one or more biometric properties over a predefined period of time by continuously comparing tissue perfusion measured on shorter channels to tissue perfusion measured on deeper channels;
determining whether the respective change satisfies one or more predetermined thresholds based on adaptive algorithms that adjust thresholds based on user-specific physiological baselines; and
responsive to determining the respective change does not satisfy the one or more predetermined thresholds, initiating one or more feedback actions.

23. The method of claim 22, wherein the NIRS system is configured to measure three or more wavelengths at three or more optical depths including wavelengths above and below an 805 nm isosbestic point, and comprises:
a weight of up to approximately 2.0 ounces;
a volume of up to approximately 30.0 cm³; and
a battery life of at least approximately 12.0 hours with continuous wireless data transmission capability.

24. The method of claim 22, wherein the one or more biometric properties comprise tissue oxygen measured at multiple anatomical depths, electrocardiography (ECG) with cardiac timing analysis, electromyography (EMG), electrooculography (EOG), pulse rate, heart rate, respiratory rate, perfusion, blood pressure, blood flow, blood volume, cardiac output, cardiac stroke volume, cardiac ejection fraction, macrovasular and microvascular hemodynamic coherence, core body temperature, or combinations thereof.

25. The method of claim 22, wherein:
monitoring the respective change comprises measuring one or more metrics from the user and one or more predetermined thresholds from the user or an environment associated with the user,
the one or more metrics include tissue oxygenation kinetics and reperfusion rates following vascular occlusion, and
the one or more metrics are associated with signal amplitude, rate, modulation depth, recovery height, change rates of individual metrics, ratios between different biometric measurements, or combinations thereof.

26. The method of claim 22, wherein the one or more feedback actions comprise a biometric property reading, one or more types of stimulation including transcranial direct current stimulation (tDCS) and transcutaneous electrical nerve stimulation (TENS), a change in an environmental property including temperature and lighting adjustments, or combinations thereof.

27. The method of claim 22, wherein the predefined period of time is associated with a duration of a medical procedure comprising an examination, testing, a pre-surgical medical observation period, a post-surgical medical observational period, a blood pressure test with vascular occlusion and reperfusion monitoring, a vascular occlusion test, a standardized physical activity test, an ortho-static test, a hyperreactivity to standing (HRS) test, a physical therapy dosing, a medication dosing, a sleep test, a resuscitation, a defibrillation, a surgical preparation, a disease progression, an injury recovery, or combinations thereof.

28. The method of claim 22, wherein the body part is a sternum of the user and the NIRS system is configured to monitor central tissue oxygenation with differentiation from peripheral measurements.

29. The method of claim 22, wherein the one or more predetermined thresholds are associated with an oxygenation level, a blood pressure reading, a vascular occlusion test output with reperfusion kinetics analysis, an ambient light exposure reading, a user motion, a sleep stage, an amount of time, previous user physiological states measured by the NIRS system, or combinations thereof.

30. The method of claim 22, further comprising:
transmitting the detected one or more biometric properties and/or the respective change to a remote device associated with a medical data system via encrypted wireless communication with real-time telemedicine integration.

31. A method of monitoring a condition of a user, the method comprising:
placing a near-infrared spectroscopy (NIRS) system on a body part of the user, wherein the NIRS system comprises a flexible substrate with integrated optical isolation and biocompatible materials;
detecting, via the NIRS system, one or more biometric properties associated with the user using spatially resolved spectroscopy (SRS) with motion artifact compensation;
monitoring a respective change associated with at least a first biometric property of the one or more biometric properties over a predefined period of time by measuring one or more metrics including tissue oxygenation kinetics and reperfusion rates, the predefined period of time being associated with a duration of a medical procedure and/or an observational period;
determining whether the respective change satisfies one or more predetermined thresholds that adapt based on user-specific physiological baselines; and
responsive to determining the respective change does not satisfy the one or more predetermined thresholds, initiating one or more feedback actions comprising real-time system parameter adjustments and environmental modifications.

32. The method of claim 31, wherein the medical procedure comprises a blood pressure test with vascular occlusion and reperfusion kinetics monitoring, a vascular occlusion test, a standardized physical activity test, an orthostatic test, a hyperreactivity to standing (HRS) test, a physical therapy dosing, a medication dosing, a sleep test, a resuscitation, a defibrillation, a surgical preparation, a disease progression, an injury recovery, or combinations thereof.

33. The method of claim 31, wherein the one or more metrics are associated with signal amplitude, rate, modulation depth, recovery height, rates of compared signal metrics, tissue oxygenation kinetics, reperfusion rates following vascular occlusion, or combinations thereof.

34. The method of claim 31, wherein the one or more feedback actions comprise a biometric property reading, one or more types of stimulation including transcranial direct current stimulation and vagus nerve stimulation, a change in an environmental property including temperature and lighting control, real-time system parameter adjustments, or combinations thereof.

35. The method of claim 31, wherein the condition of the user is associated with one or more of trauma, a disease, an injury, or combinations thereof, and the NIRS system provides continuous monitoring with predictive analytics for early detection of physiological changes.

36. A system for monitoring a condition of a user, the system comprising:
a miniaturized, ingress-protected, crush-resistant, wireless, autonomous, and self-contained near-infrared spectroscopy (NIRS) monitoring wearable system configured to be placed on a body part of the user, wherein the NIRS system comprises:
a flexible substrate comprising a sensor configured to detect one or more biometric properties associated with the user using spatially resolved spectroscopy (SRS) with both short-distance and long-distance measurements to isolate cerebral activity contributions from skin vasculature contributions,
wherein the flexible substrate is configured to conform to the body part, to provide optical isolation of NIRS optics, and to monitor a respective change associated with at least a first biometric property of the one or more biometric properties over a predefined period of time by continuously comparing tissue perfusion measured on shorter channels to tissue perfusion measured on deeper channels; and
a processor configured to:
determine whether the respective change satisfies one or more predetermined thresholds based on adaptive algorithms that adjust thresholds based on user-specific physiological baselines, and
initiate one or more feedback actions responsive to determining the respective change does not satisfy the one or more predetermined thresholds.

37. The system of claim 36, wherein the NIRS system is configured to measure three or more wavelengths at three or more optical depths including wavelengths above and below an 805 nm isosbestic point, and comprises:
a weight of up to approximately 2.0 ounces;
a volume of up to approximately 30.0 cm³; and
a battery life of at least approximately 12.0 hours with continuous wireless data transmission capability.

38. The system of claim 36, wherein the NIRS system further comprises:
the sensor configured to detect one or more optical properties of the user including different molecular states of hemoglobin comprising carboxyhemoglobin and methemoglobin; and
a non-transitory memory device configured to receive data from the sensor, the data corresponding to the one or more optical properties.

39. The system of claim 38, wherein the NIRS system further comprises:
the flexible substrate comprising biocompatible polymers and configured to provide electrical isolation;
a first light source bank capable of emitting a first set of wavelengths of red and/or near-infrared light including at least one wavelength below 730 nm and two wavelengths above the 805 nm isosbestic point, the first light source bank mounted on the substrate;
the sensor capable of detecting the first set of wavelengths with adaptive gain control, the sensor mounted on the substrate at a first distance from the first light source bank;
the processor in electronic communication with at least one of the first light source bank and the sensor, the processor mounted on the substrate and configured for real-time signal processing;
the non-transitory memory device in electronic communication with the processor, the non-transitory memory device mounted on the substrate;
a battery in electronic communication with at least one of the first light source bank, the sensor, the processor, and the non-transitory memory device; and
program instructions stored on the non-transitory memory device that, when executed, direct the processor to:
select, based on an input parameter and environmental conditions, the first set of wavelengths;
dynamically select, based on the input parameter and tissue characteristics, the first distance from the first light source bank;
process a signal from the sensor to calculate tissue oxygenation levels at multiple depths;
compare each oxygenation level to a predetermined threshold that adapts based on user-specific physiological baselines; and
at least one of:
filter the signal from the sensor using motion artifact reduction algorithms;
activate a feedback device when the oxygenation level is below the predetermined threshold and initiate real-time parameter adjustments; and
store at least one of the signal and the oxygenation level on the non-transitory memory device.

40. The system of claim 38, wherein the NIRS system further comprises:
the sensor comprising one or more detectors capable of detecting one or more biometric properties including tissue oxygenation at multiple anatomical depths; and
an electronics module communicatively and removably coupled to the substrate via wireless communication protocols and comprising:
the processor configured for parallel processing of multiple sensor inputs;
the non-transitory memory device;
an energy storage device configured to power the substrate and the electronics module with power management optimization; and
instructions stored on the non-transitory memory device that, when executed, direct the processor to:
automatically identify a type of the one or more detectors of the substrate using device recognition protocols; and
process a signal from the identified one or more detectors using machine learning algorithms to calculate one or more biometric parameters with predictive analytics.

41. The system of claim 38, wherein the NIRS system further comprises:
the flexible substrate comprising:
biocompatible materials with integrated optical isolation:
the sensor configured for multi-wavelength detection with adaptive gain; and
one or more second sensors capable of detecting one or more non-optical biometric properties of the user including electrocardiography and electromyography;
a first electronics module communicatively coupled to the substrate via encrypted wireless communication and comprising:
a first processor configured for real-time optical signal processing;
the non-transitory memory device; and
first instructions stored on the non-transitory memory device that, when executed, direct the first processor to:
receive a first signal from the sensor with motion artifact compensation;
process the first signal based on one or more first predefined algorithms; and
determine, based on the processed first signal and user-specific baselines, whether the user requires one or more first feedback actions; and
a second electronics module communicatively coupled to the substrate and comprising:
a second processor configured for multi-modal signal integration;
a second memory device; and
second instructions stored on the second memory device that, when executed, direct the second processor to:
receive a second signal from the one or more second sensors with electrical noise filtering;
process the second signal based on one or more second predefined algorithms including cardiac electrophysiology analysis; and
determine, based on the processed second signal and correlation with optical measurements, whether the user requires one or more second feedback actions;
wherein at least one of the first processor and the second processor:
responsive to at least one of the determining instructions and based on integrated multi-modal analysis,
determine that the user requires no feedback actions,
initiate at least one of the one or more first feedback actions with real-time parameter adaptation, or
initiate at least one of the one or more second feedback actions with coordinated multi-system response.
